(19) United States
(12) Patent Application Publication
(10) Pub. No.: US 2025/0186651 A1
(43) Pub. Date: Jun. 12, 2025

(54) HYDROGEL-BASED TOPICAL WOUND DRESSING CONTAINING PROTEIN EXTRACT

(71) Applicant: T&R BIOFAB CO., LTD., Siheung-si (KR)

(72) Inventors: Hyun Jung KIM, Anyang-si (KR); Chang Hwan KIM, Siheung-si (KR); Young Hwan KIM, Suwon-si (KR); Hyuk Joo AHN, Seoul (KR); Jae Hang SHIN, Siheung-si (KR)

(73) Assignee: T&R BIOFAB CO., LTD., Siheung-si (KR)

(21) Appl. No.: 19/061,366

(22) Filed: Feb. 24, 2025

Related U.S. Application Data
(63) Continuation of application No. PCT/KR2023/009483, filed on Jul. 5, 2023.

(30) Foreign Application Priority Data
Aug. 24, 2022 (KR) 10-2022-0106416

(51) Int. Cl. A61L 15/22; A61L 15/20; A61L 15/44; A61L 15/60

(52) U.S. Cl. CPC A61L 15/225; A61L 15/20; A61L 15/44; A61L 15/60; A61L 2430/10; A61L 2430/34

(57) ABSTRACT

The present invention relates to a hydrogel-based wound dressing containing a protein extract. The wound dressing comprises a polyethylene glycol, white Vaseline, 1,2-hexanediol, ethylhexylglycerin, a sodium hyaluronate, a protein extract, and purified water; maintains the same viscosity at temperatures similar to body temperature as at room temperature, thus being convenient to use; and is excellent in terms of wound healing rate, re-epithelialization rate, and blood vessel formation.

### CROSS-REFERENCE TO RELATED APPLICATIONS

This application is a Continuation of International Application No. PCT/KR2023/009483 filed Jul. 5, 2023, which claims benefit of priority to Korean Patent Application No. 10-2022-0106416 filed Aug. 24, 2022, the entire contents of which are incorporated herein by reference.

### TECHNICAL FIELD

The present disclosure relates to a hydrogel-based wound dressing containing a protein extract, and more particularly, to a topical wound dressing containing polyethylene glycol, white petrolatum, 1,2-hexandiol, ethylhexylglycerin, sodium hyaluronate, a protein extract, and purified water, which maintains a viscosity approximately equal to that of room temperature even at a temperature similar to body temperature and has an excellent wound healing effect, and a method for preparing the same.

### BACKGROUND ART

Tissue of the human skin is divided into three main parts: the epidermis, which is the outermost layer of the skin, the dermis, which is the layer underneath, and the subcutaneous tissue. Among them, the epidermis comprises epithelial cells, melanocytes, immune cells, etc. which are differentiated into several layers from the basement membrane that allows the epidermis and dermis to firmly bond, and the dermis below the epidermis mainly comprises fibroblasts, and various extracellular matrix secreted by these cells.

In general, wound healing is complex and occurs little by little in stages over time. The overlap of the three phases—hemostatic and inflammatory, proliferative, and mature—leads to the remodeling of the extracellular matrix.

In the early stages of a wound, a clot is formed due to inflammation and the reaction of the basal layer. The basal substance is a substance that fills the space between the connective fibers of the dermis and is present as a mucous mucopolysaccharide, which retains a large amount of moisture and is composed of glycosaminoglycans such as hyaluronic acid and chondroitin sulfate. In particular, proteoglycans are known to be hydrophilic carbohydrate-protein complexes and to be composed of a jelly-like substance.

Wound dressings (covering materials) are usually classified into dry dressings or moist (hydrating) dressings depending on the moisture retention of the wound. Dry dressings include bandages (adhesive bandages or other bandages) and ointments (Fucidin, Madecassol, etc.). Bandages serve to cover wounds without any therapeutic effect, and ointments contain therapeutic ingredients such as antibiotics or steroids to help wounds heal quickly and prevent further infection.

However, in the case of dry dressing, secretions such as blood and exudate dry up and disappear, leading to the loss of wound-healing cells, resulting in scarring. Additionally, when the bandage is removed, the scab comes off, increasing the risk of secondary damage. On the other hand, antibiotics contained in ointments also affect wound-healing cells, so their self-healing effect is not very high.

To compensate for the disadvantages of these dry dressings, moist dressing materials using hydrocolloid, alginate, foam, and hydrogel have been used. Among moist dressing ingredients, gelatin is a natural polymer with excellent biocompatibility, biodegradability, and high hygroscopicity, and is widely used in many wound dressings because it can provide a moist environment to the skin when a wound occurs. Hydrogel has a three-dimensional hydrophilic polymeric network structure with water as a dispersion medium, and may absorb a large amount of moisture. It also has flexibility similar to natural tissue, and is widely used in wound dressings.

However, the hydrogel patch has poor mechanical properties. In particular, the hydrogels made using collagen as the main material still has the problem of having very poor mechanical properties with a tensile strength of 0.01 to 0.05 MPa.

### DISCLOSURE

### Technical Problem

An object of the present disclosure is to provide a hydrogel-based wound dressing containing a protein extract obtained by a multi-step decellularization process, in order to effectively solve the problems of conventional wound dressing (moist dressings), and to provide a wound dressing that is convenient for users to use and has an excellent wound healing effect.

### Technical Solution

A hydrogel-based topical wound dressing material, according to one embodiment of the present disclosure, comprises polyethylene glycol (PEG), white petrolatum, 1,2-hexandiol, ethylhexylglycerin, sodium hyaluronate, a protein extract, and purified water.

The hydrogel-based topical wound dressing preferably comprises 11 to 95 wt % of polyethylene glycol, 0.5 to 10 wt % of white petrolatum, 0.1 to 10 wt % of 1,2-hexandiol, 0.01 to 10 wt % of ethylhexylglycerin, 0.001 to 10 wt % of sodium hyaluronate, 0.01 to 10 wt % of a protein extract, and residual purified water.

The polyethylene glycol (PEG) used herein is preferably a mixture of a PEG having a weight average molecular weight of 400 and a PEG having a weight average molecular weight of 4,000 in a weight ratio of about 2.2:1, and shows little change in the viscosity of hydrogel-based topical wound dressings in ointment formulations as the temperature changes from room temperature storage to human body temperature, such that the viscosity measured at 25° C. is substantially the same as the viscosity measured at 35° C.

In addition, the hydrogel-based topical wound dressing comprises a protein extract containing collagen and elastin and therefore has been shown to have excellent effects on wound healing, re-epithelialization, and angiogenesis.

Furthermore, the hydrogel-based topical wound dressing according to the present disclosure may also comprise, in place of the above 1,2-hexanediol and ethylhexylglycerin, at least one selected from the group consisting of chlorhexidine gluconate, chlorhexidine acetate, chlorhexidine hydrochloride, silver sulfadiazine, povidone iodine, benzalkonium chloride, purazine, idocaine, hexachlorophene, chlortetracycline, neomycin, penicillin, gentamicin, gentamicin sulfate, acrinol, ciprofloxacin, benzalkonium chloride, fusidic acid, neomycin sulfate, ofloxacin, and tobramycin, in an equal amount or in a range of 0.01 to 5 wt %.

The protein extract is prepared by a pretreatment step of preparing and pretreating tissue from a mammal other than a human; a first inactivation step of inactivating a virus contained in the pretreated tissue using alcohol; a decellularization step of removing cells from the virus-inactivated tissue; and a second inactivation step of inactivating a virus contained in the decellularized tissue using acid, wherein it is preferable that the DNA content of the tissue subjected to the second inactivation step is 50 ng/mg or less, and that a percentage reduction (L) of the elastin content of the tissue subjected to the pretreatment step or the second inactivation step is 20% or less.

The decellularization step may comprise a primary decellularization step of removing cells from the virus-inactivated tissue using a basic aqueous solution; and a secondary decellularization step of removing cells by enzymatically treating primary decellularized tissue.

Here, it is preferable that the primary decellularization step comprises a first decellularization step performed using a mixture of n-PrOH and NaOH; and a second decellularization step performed using an aqueous sodium hydroxide solution of more than 0.05 M and less than 0.2 M.

It is preferable that the tissue is at least one of mammal-derived blood vessels, ligaments, and tendons, and it is more preferable that the secondary decellularization step is performed using DNase.

### Advantageous Effects

The hydrogel-based topical wound dressing containing a protein extract according to the present disclosure does not cause toxicity problems in human body, has excellent biocompatibility, and is convenient to use because its viscosity does not decrease despite temperature changes when applied to the wounded skin tissue by the use.

In addition, the protein extract obtained by multi-step decellularization and inactivation steps has excellent wound healing effects, re-epithelialization, and angiogenesis, and may therefore be used as a useful wound dressing for the treatment of wounds.

### DESCRIPTION OF DRAWINGS

FIGS. 1A and 1B are graphs showing the experimental results of Experimental Example 1, respectively.

FIG. 2 is a graph showing the experimental results of Experimental Example 2.

FIGS. 3A and 3B are graphs showing the experimental results of Experimental Example 3.

FIGS. 4A and 4B are graphs showing the experimental results of Experimental Example 4.

FIG. 5 is a table showing the experimental results of Experimental Example 5.

FIGS. 6A to 6D show the results of measuring the viscosity, storage modulus, and pH, which are the basic properties of the topical wound dressing according to Preparation Example 2.

FIG. 7 shows the results of measuring the cell proliferation rate using Cell Count Kit-8.

FIGS. 8A and 8B show the results of measuring the cell viability in the hydrogel using Live/Dead assay kit (Invitrogen).

FIGS. 9A and 9B show the results of a scratch evaluation performed on the eluted culture solution.

FIG. 10 is a schematic representation of an acute wound animal model in which Experimental Example 8 was performed.

FIGS. 11A to 13B are the results of wound healing rate (%), re-epithelialization rate (%), or angiogenesis (%) according to Experimental Example 8, respectively.

### BEST MODE

Before describing the present disclosure in more detail below with reference to preferred embodiments of the present disclosure, it should be noted that the terms and words used in this specification and claims are not to be construed in their ordinary or dictionary sense, but rather in a sense and concept consistent with the technical ideas of the present disclosure.

Throughout this specification, “comprising” any component will be understood to imply the inclusion of other components rather than the exclusion of other components, unless otherwise specifically stated.

Throughout the present specification, “%” used to indicate a concentration of a particular substance refers to (weight/weight) % for solid/solid, (weight/volume) % for solid/liquid, and (volume/volume) % for liquid/liquid, unless otherwise stated.

In addition, throughout the present specification, “mammal” refers to a mammal other than a human, and should be understood as a mammal other than a human, even if it is simply stated as “mammal” without any reference to excluding a human. The expression “cells are removed” should be understood in a comprehensive sense to include the destruction or removal of DNA.

In addition, the steps may be performed in any order other than the order specified unless the context clearly indicates a particular order. That is, the steps may be performed in the same order as specified, substantially simultaneously, or in reverse order.

Further, unless otherwise defined, all technical and scientific terms used herein shall have the same meanings as commonly understood by those skilled in the art to which the present disclosure belongs, and in the case of conflict, the description in the present specification, including definitions, will prevail.

A hydrogel-based wound dressing containing a protein extract according to one embodiment of the present disclosure is a composition effective for wound healing for use on wounded skin tissue in an ointment formulation, comprising 11 to 95 wt % of polyethylene glycol, 0.5 to 10 wt % of white petrolatum, 0.1 to 10 wt % of 1,2-hexandiol, 0.01 to 10 wt % of ethylhexylglycerin 0.001 to 10 wt % of sodium hyaluronate 0.01 to 10 wt % of a protein extract, and the residual purified water, and may provide a topical wound dressing that maintains the same viscosity at temperatures similar to body temperature as at room temperature, and has excellent wound healing, re-epithelialization, and angiogenesis.

Hydrogel refers to a three-dimensional network structure made by cross-linking hydrophilic polymers with covalent or non-covalent bonds and due to the hydrophilicity of the constituent materials, it absorbs a large amount of water in an aqueous solution and in an aqueous environment and swells, but has a property of not dissolve due to the cross-linked structure. In addition, hydrogels with various shapes and properties may be produced depending on the components and preparation method, and may contain a large amount of moisture, giving them intermediate properties between liquid and solid.

The topical wound dressing of the present disclosure, implemented in an ointment formulation, is prepared based on a hydrogel containing polyethylene glycol (PEG) as a main component, unlike other existing wound dressings of the white petrolatum series, and thus has excellent mechanical properties. In particular, polyethylene glycol (PEG) may be used as a mixture of PEG having a weight average molecular weight of 400 and PEG having a weight average molecular weight of 4,000 in a weight ratio of about 2.2:1, or may be used in a range of 10 to 95 wt % of PEG having a weight average molecular weight of 400 and 1 to 25 wt % of PEG having a weight average molecular weight of 4,000, based on 100 wt % of the topical wound dressing.

By using a PEG mixture having such different molecular weights, the mechanical properties of the topical wound dressing used in the ointment formulation are excellent, and the viscosity at the storage temperature of room temperature (about 25° C.) and the viscosity at the temperature of the skin where the topical wound dressing is used (about 36° C.) are substantially the same with almost no change, so when a user uses the topical wound dressing according to the present disclosure on a wounded area, it does not easily flow down but is fixed around the wound, thereby improving the convenience of the user.

Furthermore, in order to further enhance the wound healing effect, the topical wound dressing according to the present disclosure comprises a protein extract containing collagen and elastin in a range of about 0.01 to 10 wt %. The protein extract is obtained by a multi-step decellularization process, and has effectively removed cells, crude fat, viruses, and other foreign substances from mammalian-derived tissues, with minimal loss of elastin during the decellularization process. It demonstrates excellent effects in re-epithelialization and angiogenesis.

In addition, the topical wound dressing according to the present disclosure may further comprise white petrolatum, 1,2-hexandiol, ethylhexylglycerin, and sodium hyaluronate, which act as contact and preservative agents on the skin or wound area. Preferably, the aforementioned mixture of 11 to 95 wt % of polyethylene glycol and 0.01 to 10 wt % of a protein extract may be mixed with 0.5 to 10 wt % of white petrolatum, 0.1 to 10 wt % of 1,2-hexandiol, 0.01 to 10 wt % of ethylhexylglycerin, 0.001 to 10 wt % of sodium hyaluronate, and the residual purified water.

Hyaluronic acid is a type of polysaccharide in which repeating units composed of N-acetyl-glucosamine and D-glucuronic acid are linearly linked, and is a biopolymer. It was first isolated from the fluid filling the eyeballs of animals, and it has been known to be present in various animal tissues such as placentas, synovial fluid in joints, pleural fluid, skin, and comb of roosters. It is also produced by microorganisms of the genus Streptococcus, such as Streptococcus equi and Streptococcus zooepidemecus.

Hyaluronic acid has excellent biocompatibility and high viscoelasticity in solution, and is widely used not only for cosmetic applications such as cosmetic additives, but also for various medicinal applications such as ophthalmic surgical aids, joint function enhancers, drug delivery substance, and eye drops. However, hyaluronic acid itself is limited in its use because it is easily degraded in vivo or under conditions such as acids and alkalis.

Meanwhile, the hydrogel-based topical wound dressing according to the present disclosure may comprise, in place of the aforementioned 1,2-hexanediol and ethylhexylglycerin, at least one other selected from the group consisting of chlorhexidine gluconate, chlorhexidine acetate, chlorhexidine hydrochloride, silver sulfadiazine, povidone iodine, benzalkonium chloride, purazine, idocaine, hexachlorophene, chlortetracycline, neomycin, penicillin, gentamicin, gentamicin sulfate, acrinol, ciprofloxacin, benzalkonium chloride, fusidic acid, neomycin sulfate, ofloxacin, and tobramycin, in an amount equal to that of 1,2-hexanediol and ethylhexylglycerin that can be used, or in an amount of about 0.01 to 5 wt % in the total composition.

Hereinafter, the protein extract component obtained through the multi-step decellularization process will be described in more detail.

The protein extract contained in a topical wound dressing according to the present disclosure may be obtained by a preparation step of preparing tissue from a mammal, other than a human; a pretreatment step of pretreating the tissue; a first inactivation step of inactivating a virus contained in the pretreated tissue using alcohol; a primary decellularization step of removing cells from the virus-inactivated tissue using a basic aqueous solution; a secondary decellularization step of removing cells by enzymatically treating the primary decellularized tissue; and a second inactivation step of inactivating a virus contained in the decellularized tissue using an acid.

In this case, the DNA content of the tissue subjected to the second inactivation step may be 50 ng/mg or less, preferably 20 ng/mg or less, 10 ng/mg or less, more preferably 5 ng/mg or less. Further, the percentage reduction (L) of the elastin content of the tissue subjected to the pretreatment step or the second inactivation step is 20% or less.

Herein, the percentage reduction (L) is defined by Equation (1) below, where L₀ means the elastin content of the tissue in the preparation step, and L_{f} means the elastin content of the tissue subjected to the second inactivation step:

$$L ( \% ) = \frac{( {L}_{0} - {L}_{f} )}{{L}_{0}} \times 100. \qquad ( Equation 1 )$$

Firstly, the above preparation step is a step of preparing tissue from a mammal other than a human. Here, the mammal may be a mammal other than a human, for example, a pig, a horse, a cow, a sheep, etc., and the tissue may be at least one of such mammal-derived blood vessels, ligaments, and tendons.

In order to prepare a multi-step decellularized protein extract, tissue from a mammal is first harvested. The tissue from a mammal thus harvested is prepared by removing any unwanted tissue and blood that adheres to the tissue from a mammal in the preparation step, and by washing, drying, and cutting the obtained tissue. The tissue from a mammal thus prepared may be stored in a frozen state and may be thawed and used when required for use. If tissue materials in a frozen state is used, the preparation step may be a step of taking out and preparing the tissue materials in a frozen state.

Next, the tissue from a mammal prepared in the preparation step is pretreated through a pretreatment step.

This step is a step of washing the tissue from a mammal. If the tissue from a mammal is frozen, it may be thawed, washed, and cut through this step. In this step, distilled water, purified water, saline, etc., may be used as washing water, preferably distilled water. Physical stirring may be performed from this step to the second inactivation step.

The tissue from a mammal thus prepared through the pretreatment steps may undergo a first inactivation step, a primary decellularization step, a secondary decellularization step, and a second inactivation step to remove cells, crude fat, viruses, and other foreign substances that may induce immune and foreign body reactions. Decellularization through such a multi-step reaction significantly improves the decellularization efficiency and foreign substance removal efficiency at each step, thus increasing the amount of tissue processed at a time, shortening the processing time, and consequently improving the productivity and yield of the decellularized protein extract.

First, the first inactivation step is a step of inactivating a virus contained in the pretreated tissue using alcohol.

This step is performed to more effectively remove cells, crude fat, and foreign substances during the primary and secondary decellularization steps by primarily inactivating the virus in the tissue before decellularizing the tissue.

The alcohol used in this step may be n-propanol, and preferably an aqueous n-propanol solution having a concentration of 50 to 90%, more preferably an aqueous n-propanol solution having a concentration of 65 to 80% may be used to achieve a virus inactivation effect.

In the first inactivation step, n-propanol is used as alcohol for virus inactivation. In these cases where n-propanol is used for virus inactivation, the tissue is softer, allowing for better penetration of the treatment solution into the tissue, and thus resulting in uniform virus inactivation and decellularization throughout. As a result, the treatment efficiency of the inactivation and decellularization may be improved, more homogeneous tissue may be obtained, and the quality and reliability of the finally obtained decellularized protein extract may be improved.

Generally, when ethanol is used for virus inactivation, it has the characteristic of fixing and hardening the tissue of a mammal to be decellularized. This is undesirable if ethanol is used instead of n-propanol at this step of the present disclosure, as it hardens the tissue, reducing the efficiency of the process to improve the penetration of the treated substance by loosening the tissue in subsequent steps.

Furthermore, since iso-propanol has a lower viral inactivation effect, when iso-propanol is used in the first inactivation step, a sufficient viral inactivation effect is not obtained, resulting in reduced decellularization efficiency in the subsequent primary and secondary decellularization steps; therefore, n-propanol is preferably used in the first inactivation step.

In this step, the pretreated tissue may be treated with 10 to 25 parts by weight, preferably 20 to 25 parts by weight, relative to 100 parts by weight of the propanol aqueous solution.

The primary decellularization step is a step of removing cells by treating the virus-inactivated tissue with a basic aqueous solution through the first inactivation step.

This step first comprises a first decellularization step of treating the tissue using a mixture of a basic aqueous solution and an alcohol; and a second decellularization step of treating the tissue using a basic aqueous solution. Here, the basic aqueous solution is an aqueous sodium hydroxide solution, and the alcohol is preferably n-propanol. In this case, the first decellularization step is performed by treating the tissue with a mixture of n-propanol and sodium hydroxide, and the second decellularization step is performed by treating the tissue with an aqueous sodium hydroxide solution.

When the decellularization process is performed in two steps, the first decellularization step not only removes the detached cells but also removes foreign substances such as crude fat, allowing for a more effective decellularization process in the second decellularization step.

In each of the first and second decellularization steps, the tissue may be treated in an amount of 10 to 25 parts by weight, preferably 20 to 25 parts by weight, relative to 100 parts by weight of the treatment solution, which is nearly twice a conventional treatment capacity. Since effective decellularization is possible through the multi-step method of the present disclosure, there is an effect that allows for sufficient treatment of twice the amount of tissue compared to the conventional method.

Specifically, the first decellularization step is a step of treating the virus-inactivated tissue with a mixture of n-propanol and sodium hydroxide. In this step, the crude fat and sodium hydroxide in the tissue undergo a saponification reaction, and the crude fat is separated and removed from the tissue. Here, n-propanol promotes this saponification reaction and acts to cause the products of the saponification reaction to coagulate with each other, thereby allowing the crude fat to be separated and removed from the tissue more quickly and efficiently.

Here, the treatment time for treating the tissue with a mixture of n-propanol and sodium hydroxide may be 10 to 48 hours, preferably 15 to 40 hours, and more preferably 18 to 28 hours, and the treatment temperature may be 0 to 30° C., preferably 10 to 28° C., and more preferably 18 to 25° C. When treated within these temperature ranges for the above time, sufficient crude fat removal efficiency may be obtained without significant damage of the tissue structure or loss of elastin.

The mixture of n-propanol and sodium hydroxide may be an aqueous solution having a concentration of n-propanol of 50 to 95%, preferably 60 to 90%, and more preferably 65 to 85%, and a concentration of sodium hydroxide of more than 0.05 M and less than 0.2 M. These concentration ranges may minimize tissue damage in the first decellularization step while effectively removing foreign substances such as crude. In particular, if the concentration of sodium hydroxide is below the above range, the treatment efficiency of the first decellularization step decreases, and if the concentration of sodium hydroxide is outside the above range, the tissue structure itself collapses and dissolves. Therefore, it is preferable to use n-propanol and sodium hydroxide aqueous solutions within the above-mentioned concentration ranges.

The second decellularization step is a step wherein the tissue subjected to the first decellularization step is treated with a basic aqueous solution to soften the tissue, thereby loosening the structure of the tissue and simultaneously causing a decellularization reaction. The crude fats and various foreign substances contained in the tissue act as a kind of physical and chemical barrier to the decellularization process, and since the tissues have already undergone the first decellularization step and the crude fat and various foreign substances have been removed, the decellularization reaction may be more effectively preformed in the second decellularization step.

In this step, the treatment time for treating the tissue in the basic aqueous solution may be 10 to 48 hours, preferably 15 to 40 hours, and more preferably 18 to 28 hours, and the treatment temperature may be 0 to 30° C., preferably 10 to 28° C., and more preferably 18 to 25° C. When treated within these temperature ranges for the above time, the tissue structure may be sufficiently loosened and deformed without collapse of the tissue structure or loss of elastin, allowing for an appropriate decellularization reaction to occur.

In this case, the basic aqueous solution used as the treatment solution may be an aqueous sodium hydroxide solution, and the concentration of sodium hydroxide contained in the aqueous sodium hydroxide solution may be more than 0.05 M and less than 0.2 M. If the concentration of sodium hydroxide is 0.05 M or less, the tissue structure may not be sufficiently loosened to allow for efficient decellularization in the secondary decellularization step described later. If the concentration of sodium hydroxide is 0.2 M or more, some tissue may be collapsed or dissolved beyond the level of loosening in this step, resulting in a significant reduction in final yield and loss of elastin. Thus, it is preferable to use an aqueous sodium hydroxide solution of the above-mentioned centration.

After the primary decellularization step, a secondary decellularization step using an enzyme may be performed, and a neutralization step and a washing step may be performed between these two steps.

The neutralization step is a step of treating with an acidic aqueous solution to neutralize the sodium hydroxide used in the primary decellularization step, wherein the type of acidic aqueous solution used may be, for example, but is not limited to, an acidic aqueous solution containing at least one of hydrochloric acid, sulfuric acid, acetic acid, peracetic acid, and the concentration thereof may also be appropriately adjusted and used according to the working environment or conditions.

The washing step is performed to prevent the enzyme reactivity from being reduced due to residues during enzymatic treatment in the subsequent secondary decellularization step. A buffer solution may be used as the washing solution used in the washing step, for example, a phosphate buffered saline (PBS) solution may be used, but is not limited thereto.

Meanwhile, the secondary decellularization step is a step of removing enzymatically treating the primary decellularized tissue, and a step of treating the primary decellularized tissue with a DNA-degrading enzyme to remove DNA in the primary decellularized tissue.

In this step, the tissue may be treated with 10 to 25 parts by weight, preferably 20 to 25 parts by weight, relative to 100 parts by weight of the treatment solution containing the DNA-degrading enzyme, which is a treatment capacity that is nearly twice that of the conventional treatment as described above, and this is an achievable treatment capacity because of increased treatment efficiency provided by the multi-step decellularization process of the present disclosure.

In this step, in order to obtain enzyme activity and sufficient DNA degradation efficiency, the treatment time for treating the tissue with the DNA-degrading enzyme may be 10 to 35 hours, preferably 18 to 30 hours, and the treatment temperature may be 30 to 45° C., preferably 35 to 42° C.

In the present disclosure, the concentration of DNA-degrading enzyme contained in the treatment solution is 0.0001 to 0.005 wt %, which is several times to several hundred times lower than the concentration of DNA-degrading enzyme typically used in similar techniques in the prior art. The primary decellularized tissue of the present disclosure is subjected to a primary decellularization step, and foreign substances containing crude fat in the tissue are removed and the structure of the tissue is loosened. In this state, when the DNA-degrading enzyme is introduced, it is very easy for the DNA-degrading enzyme to penetrate into the tissue structure, and accordingly, at least the same or better DNA removal efficiency may be secured even if a low concentration of DNA-degrading enzyme is used, so that a low concentration of DNA-degrading enzyme as described above may be used in the present disclosure.

However, if the concentration of the DNA-degrading enzyme is below the above range, the DNA degradation efficiency decreases, so it is preferable to use a concentration of 0.0001% or more. If the concentration of the DNA-degrading enzyme exceeds the above range, it is uneconomical to use the DNA-degrading enzyme in the above concentration range because the improvement in DNA degradation efficiency is extremely small compared to the amount of DNA-degrading enzyme added and thus is preferable to use the DNA-degrading enzyme of the above-mentioned concentration range.

The decellularized tissue subjected to the secondary decellularization step is subjected to a second inactivation step to further remove the virus, and an additional decolorization and/or delipidation step may be performed between these two steps. The decolorization step is a step of removing the color of the tissue, which may be performed, for example, by treating the tissue with hydrogen peroxide. The delipidation step is a step of further removing the fat contained in the tissue, which may be performed, for example, using a ketone solution, preferably an acetone solution.

Then, a second inactivation step is performed to additionally inactivate the virus contained in the tissue that has undergone the secondary decellularization step. This step is a step to inactivate the virus in the tissue by using acid, specifically, a step to treat the tissue with a mixture of organic acids and alcohol.

The organic acid used at this time may be peracetic acid, which is an oxidizing agent and may inactivate the virus in a manner that causes unspecified free radical damage to the virus. The peracetic acid used in this step may be used at a concentration of 0.05 to 1%.

Furthermore, the alcohol used in this step may be a lower alcohol with a carbon number of 1 to 4, such as methanol, ethanol, or propanol, and preferably ethanol. It is preferable to use ethanol because ethanol has the function of inactivating viruses and fixing tissue structures to make the tissues firm.

Through these steps, the virus is finally inactivated, the crude fat and DNA are removed, and a protein extract containing various extracellular matrix components containing elastin may be obtained.

The content of DNA contained in the protein extract thus obtained may be 50 ng/mg or less, preferably 20 ng/mg or less, 10 ng/mg or less, and even more preferably 5 ng/mg or less, such that the induction of immune or various foreign body reactions may be minimized by having a very low DNA content.

Furthermore, the protein extract has the advantage of having a very low elastin content reduced by the above pretreatment step or the second inactivation step, thereby having effects such as wound healing and scar suppression by elastin. Specifically, the percentage reduction (L) of the elastin content of the tissue subjected to the pretreatment step or the second inactivation step is 20% or less. These numerical values are very low compared to the very high percentage reduction (L) of elastin content of 50% or more when conventional decellularization methods are applied.

Meanwhile, after the second inactivation step, additional steps such as washing, packaging, sterilization, and processing may be performed. Washing may be performed using a buffer solution, distilled water, etc., and packaging may be performed to prevent contamination and facilitate handling when storing or transporting the protein extract, and may be packaged in a state of being immersed in a preservative solution.

Sterilization may be performed to remove additional contamination that occurs after the second inactivation step, and for sterilization, for example, electron beam irradiation or radiation may be performed, but is not limited thereto.

Processing is a process of processing the above protein extract into a usable form, and for example, methods such as drying, powdering, and solutionization may be applied. It may be utilized in the form such as bioink or injection by being processed as described above, and is not limited thereto and may be utilized in various forms.

Meanwhile, the method for preparing these protein extracts will be described in more detail as follows, and the overlapped matter with those already described above are omitted.

The method for preparation comprises a preparation step of preparing tissue from a mammal other than a human; a pretreatment step of pretreating the tissue; a first inactivation step of inactivating a virus contained in the pretreated tissue using alcohol; a primary decellularization step of removing cells from the virus-inactivated tissue using a basic aqueous solution; a secondary decellularization step of removing cells by enzymatically treating the primary decellularized tissue; and a second inactivation step of inactivating a virus contained in the decellularized tissue using an acid.

Here, the DNA content of the tissue subjected to the second inactivation step may be 50 ng/mg or less, and the percentage reduction (L) of the elastin content of the tissue subjected to the pretreatment step or the second inactivation step may be 20% or less.

Here, the percentage reduction (L) of the elastin content of the tissue is defined by Equation (1) below, where L₀ is the elastin content of the tissue in the preparation step, and L_{f} is the elastin content of the tissue subjected to the second inactivation step:

$$L ( \% ) = \frac{( {L}_{0} - {L}_{f} )}{{L}_{0}} \times 100. \qquad ( Equation 1 )$$

Firstly, the above preparation step is a step of preparing the tissue from a mammal other than a human. Here, the mammal may be a mammal other than a human, for example, a pig, a horse, a cow, a sheep, etc., and the tissue may be at least one of such mammal-derived blood vessels, ligaments, and tendons.

The pretreatment step is a step of washing the tissue from a mammal prepared in the preparation step, wherein the tissue may be cut to an appropriate size at this step, if necessary, and if the tissue from a previously frozen state is prepared, thawing may be performed at this step.

The first inactivation step is a step of inactivating a virus contained in the pretreated tissue using alcohol. Generally, ethanol is used for virus inactivation, but in the present disclosure, n-propanol is used. This is because ethanol firmly fixes the tissue to reduce the efficiency of softening in subsequent steps, whereas n-propanol softens the tissue to improve the efficiency of softening in subsequent steps.

The primary decellularization step is a step of removing cells by treating the virus-inactivated tissue with a basic aqueous solution through the first inactivation step. The step first comprises a first decellularization step of treating the tissue using a mixture of a basic aqueous solution and an alcohol; and a second decellularization step of treating using a basic aqueous solution.

Here, the basic aqueous solution is an aqueous sodium hydroxide solution, and the alcohol is preferably n-propanol.

Specifically, the first decellularization step is a step of treating the virus-inactivated tissue with a mixture of n-propanol and sodium hydroxide, and through this step, the crude fat and foreign substances in the tissue are removed and the tissue is partially softened. The second decellularization step is a step of treating the tissue with an aqueous sodium hydroxide solution, wherein the decellularization reaction occurs simultaneously with the softening of the tissue, and since the foreign substances containing the crude fat may be removed, such an effect may be further enhanced.

In the first and second decellularization steps, sodium hydroxide may be included in a concentration of more than 0.05 M and less than 0.2 M, which is a desirable concentration range for sufficient saponification and softening reactions by sodium hydroxide while preventing tissue disintegration or dissolution.

Subsequently, after the primary decellularization step, a secondary decellularization step may be performed using an enzyme, and a neutralization step and a washing step may be performed between these two steps. The neutralization step is the step of treating with an acidic aqueous solution to neutralize the sodium hydroxide used in the primary decellularization step, and the washing step is the step performing to prevent the enzyme reactivity from being reduced due to residues during enzymatic treatment in the subsequent secondary decellularization step.

The secondary decellularization step is a step removing DNA in the tissue by treating the primary decellularized tissue with a DNA-degrading enzyme. The primary decellularized tissue of the present disclosure is subjected to a primary decellularization step, and foreign substances containing the crude fat in the tissue are removed and the structure of the tissue is loosened. In this state, when the DNA-degrading enzyme is introduced, it is very easy for the DNA-degrading enzyme to penetrate into the tissue structure, and accordingly, at least the same or better DNA removal efficiency may be obtained even if a low concentration of DNA-degrading enzyme is used, so that the DNA-degrading enzyme in the present disclosure may be used at a low concentration of 0.0001 to 0.005 wt %. If the concentration of the DNA-degrading enzyme is below the above range, the DNA degradation efficiency decreases, and if the concentration of the DNA-degrading enzyme exceeds the above range, it is uneconomical to use the DNA-degrading enzyme in the above concentration range because the improvement in DNA degradation efficiency is extremely small compared to the amount of DNA-degrading enzyme added and thus is preferable to use the DNA-degrading enzyme within the above-mentioned concentration range.

The decellularized tissue subjected to the second decellularization step is subjected to a second inactivation step to further remove the virus, and an additional decolorization and/or delipidation step may be performed between these two steps. The decolorization step a step of removing the color of the tissue, which may be performed, for example, by treating the tissue with hydrogen peroxide. The delipidation step is a step of further removing the fat contained in the tissue, which may be performed, for example, using a ketone solution, preferably an acetone solution.

Then, a second inactivation step is performed to additionally inactivate the virus contained in the tissue that has undergone the secondary decellularization step. This step is a step of treating the tissue with a mixture of organic acids and alcohols, wherein peracetic acid may be used as the organic acid, and ethanol may be used as the alcohol, and by treating the tissue using this mixed solution, the virus in the tissue may be effectively removed.

Through these steps, the virus is finally inactivated, the crude fat and DNA are removed, and a protein extract containing various extracellular matrix components containing elastin may be prepared.

The content of DNA contained in the protein extract thus obtained may be 50 ng/mg or less, preferably 20 ng/mg or less, 10 ng/mg or less, and even more preferably 5 ng/mg or less, such that the induction of immune or various foreign body reactions may be minimized by having a very low DNA content.

Furthermore, the protein extract has the advantage of having a very low elastin content reduced by the above pretreatment step or the second inactivation step, thereby having effects such as wound healing and scar suppression by elastin. Specifically, the percentage reduction (L) of the elastin content of the tissue subjected to the pretreatment step or the second inactivation step is 20% or less. These numerical values are very low compared to the very high percentage reduction (L) of elastin content of 50% or more when conventional decellularization methods are applied.

Meanwhile, after the second inactivation step, additional steps such as washing, packaging, sterilization, and processing may be performed. Washing may be performed using a buffer solution, distilled water, etc., packaging may be performed to prevent contamination and facilitate handling when storing or transporting the protein extract, and sterilization may be performed to remove additional contamination that occurs after the second inactivation step.

Processing is a process of processing the above protein extract into a usable form, and for example, methods such as drying, powdering, and solutionization may be applied. It may be utilized in the form such as bioink or injection by being processed as described above, and is not limited thereto and may be utilized in various forms.

Hereinafter, specific operations and effects of the present disclosure will be described with reference to one embodiment of the present disclosure. However, this is presented as a preferred example of the present disclosure, and the scope of the present disclosure is not limited by the Examples.

### Preparation Example 1

### 1. Preparation and Pretreatment Steps

First, porcine heart connective aorta, which was washed, dried, cut, and frozen, was prepared, thawed, washed with distilled water, and then cut into 50×50 mm sizes to prepare raw tissue specimens.

### 2. First Inactivation Step

Next, 250 g of raw tissue specimens were added to 1 L of 70% n-propanol and stirred at a temperature of 20° C. and a stirring speed of 150 RPM for 24 hours to perform a first inactivation step.

### 3-1. Primary Decellularization (First Step Decellularization Step)

Then, a mixture solution containing 70% n-propanol and 0.1 M sodium hydroxide in distilled water was prepared, and 23 parts by weight of the raw tissue specimen was mixed, relative to 100 parts by weight of the mixed solution, and then stirred at a temperature of 20° C. and a stirring speed of 150 RPM for 24 hours to perform a first decellularization step.

### 3-2. Primary Decellularization Step (Second Decellularization Step)

Next, the tissue specimens subjected to the first decellularization step was mixed with 24 parts by weight, relative to 100 parts by weight of a previously prepared 0.1 M aqueous sodium hydroxide solution, and then stirred for 24 hours under the same conditions as the previous step to perform a second decellularization step.

Then, tissue specimens subjected to the second decellularization step were immersed in an aqueous acetic acid solution, stirred, neutralized, and then washed with a PBS solution.

### 4. Secondary Decellularization Step

Then, the tissue specimens were placed in a treatment solution containing 0.00011 wt % DNase and stirred at a stirring speed of 150 RPM at a temperature of 37° C. for 23 hours to perform a secondary decellularization step. The tissue specimens that had completed the second decellularization step were decolorized by treating them in 3% hydrogen peroxide solution at 20° C. for 1 hour, and degreased by treating them in 20% acetone aqueous solution at 20°0 C. for 1 hour.

### 5. Second Inactivation Step

Then, the tissue specimens were immersed in a mixed solution containing 70% n-propanol and 0.2% peracetic acid in distilled water, and treated at 20° C. for four and a half hours to perform a second inactivation step.

Finally, the tissue specimens subjected to all of the above processing steps were washed with a PBS solution and distilled water to prepare a protein extract contained in the topical wound dressing of the present disclosure.

### Experimental Example 1

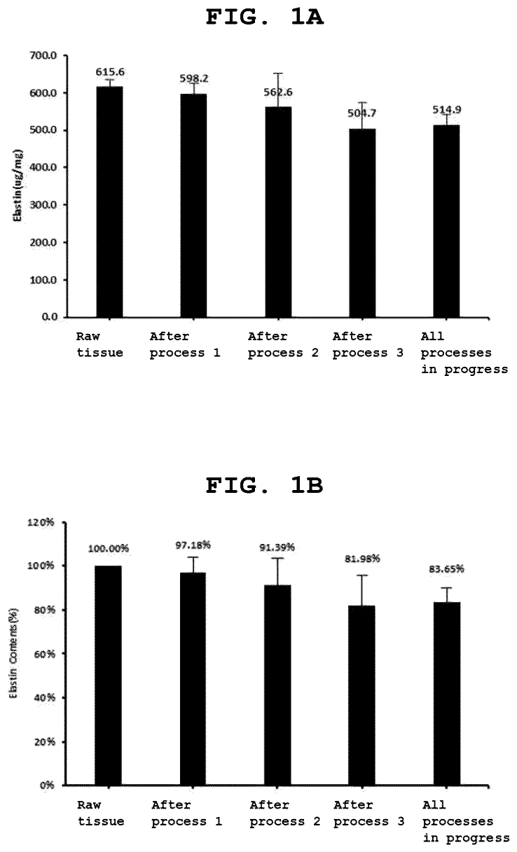

A multi-step decellularized protein extract was prepared using the same method as in Preparation Example 1, but when the first, second, and secondary decellularization steps were performed sequentially, the tissue specimens were collected immediately after each step was performed, and the elastin content per 1 mg of the tissue specimen was measured, and the results are shown in FIG. 1A. In addition, the elastin content of the raw tissue was measured at the preparation step, and the elastin content at the time of each step was performed was expressed as a percentage with respect to the elastin content of the raw tissue, and was shown in FIG. 1B.

In FIGS. 1A and 1B, process 1 refers to a first decellularization step, process 2 refers to a second decellularization step, and process 3 refers to a second decellularization step.

The experiment was performed using Fastin Elastin Assay kit F2000 (manufacturer: Biocolor) as the assay kit according to the protocol of the assay kit, and then the elastin was analyzed at an absorbance of 513 mm using a multimode plate reader Victor Nivo™ (manufacturer: Perkin Elmer), and the elastin content was measured by comparing it with a standard material.

Referring to the results in FIGS. 1A and 1B, it can be confirmed that the elastin content is slightly lost as each process progresses, but at the end of the final step, about 80% or more elastin remains.

### Experimental Example 2

A multi-step decellularized protein extract was prepared using the same method as in Preparation Example 1, but each of the first, second, and secondary decellularization steps was omitted by one step, the tissue specimens subjected to the final process was collected, and the content of elastin per 1 mg of the tissue specimens was measured. In the preparation step, the elastin content of the raw tissue was measured by the same method, and the L value was calculated and the results were summarized in Table 1.

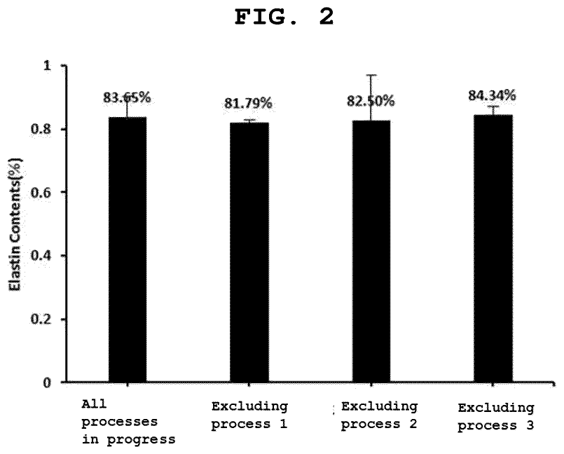

In addition, the elastin content of the raw tissue in the preparation step was calculated as a percentage of the elastin content contained in the protein extract obtained by omitting each step, and the results were shown in FIG. 2.

The elastin content was measured in the same manner as in Experimental Example 1, wherein in Table 1 and FIG. 2, process 1 refers to a first decellularization step, process 2 refers to a second decellularization step, and process 3 refers to a secondary decellularization step.

TABLE 1

| | | | |
|---|---|---|---|
| All | | | |
| processes | Excluding | Excluding | Excluding |
| in progress | process 1 | process 2 | process 3 |

| | |
|---|---|
| L₀ | 615.6 |

| | | | | |
|---|---|---|---|---|
| (μg/mg) | | | | |
| L_{f} | 514.9 | 503.5 | 507.8 | 519.1 |
| (μg/mg) | | | | |
| L (%) | 16.4 | 18.2 | 17.5 | 15.7 |

First, referring to Table 1, it can be confirmed that the L value is 20% or less both when all processes are performed and when one process is omitted. Referring to FIG. 2, it can be confirmed that the elastin content does not change significantly even when a specific process is omitted. Therefore, from these experimental results, it can be confirmed that even if any one of the first decellularization step, the second decellularization step, and the secondary decellularization step is omitted in the process according to one embodiment of the present disclosure, the elastin content is not significantly affected.

### Experimental Example 3

The experiment was conducted in the same manner as in Experimental Example 1, but the DNA content, rather than the elastin content, was measured after performing each step. The experiment was performed using Quant-iT™ PicoGreen™ dsDNA Assay kits as the assay kit and dsDNA Reagents (manufacturer: Invitrogen) according to the protocol of the assay kit, and then DNA content was measured at an absorbance of 480 to 520 mm using a multimode plate reader Victor Nivo™ (manufacturer: Perkin Elmer).

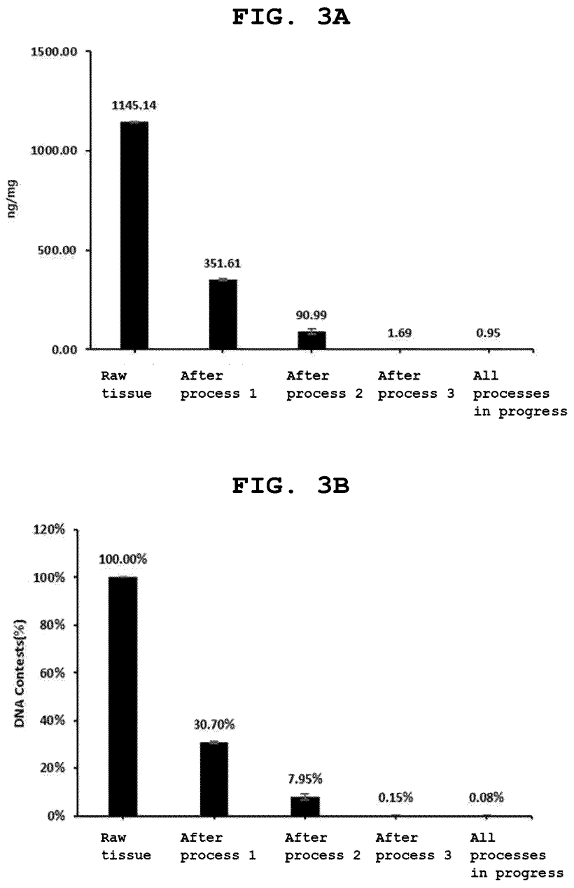

After performing each step, the DNA content per 1 mg of tissue is shown in FIG. 3A, and the DNA content of the tissue after performing each step relative to the DNA content of the raw tissue is shown in FIG. 3B as a percentage value. At this time, in FIGS. 1A and 1B, process 1 refers to a first decellularization step, process 2 refers to a second decellularization step, and process 3 refers to a secondary decellularization step.

Referring to FIGS. 3A and 3B, it can be confirmed that most of the DNA is removed after subjecting to all steps of the first decellularization step, the second decellularization step, and the secondary decellularization step.

### Experimental Example 4

The experiment was conducted in the same manner as in Experimental Example 2, but each step was omitted, and the DNA content of the prepared protein extract was measured in the same manner as in Experimental Example 3.

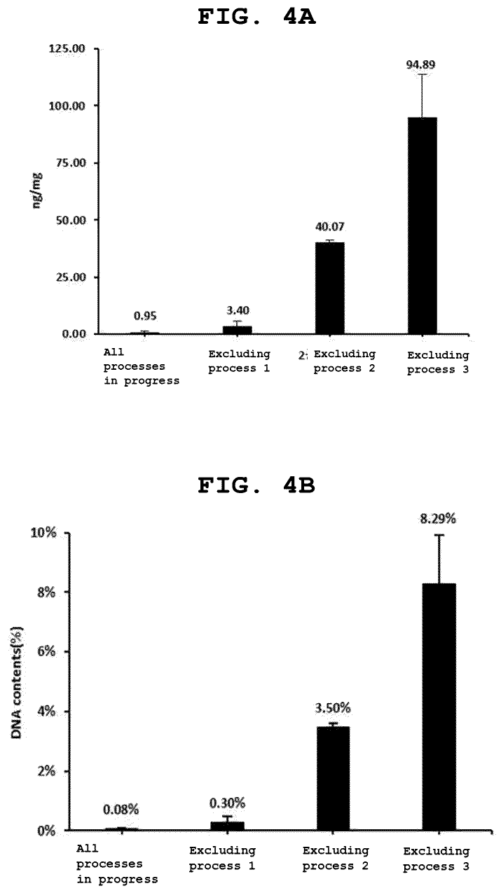

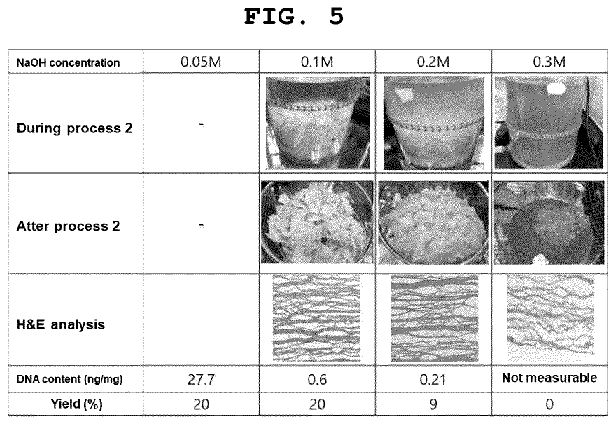

The DNA content per 1 mg of protein extract obtained by omitting each step is shown in FIG. 4A, and the DNA content of the tissue obtained by omitting each step relative to the DNA content of the raw tissue is shown in FIG. 4B as a percentage value. At this time, in FIGS. 4A and 4B, process 1 refers to a first decellularization step, process 2 refers to a second decellularization step, and process 3 refers to a secondary decellularization step.

Referring to FIGS. 4A and 4B, it can be confirmed that most of the DNA is clearly removed only after subjecting to all steps of the first decellularization step, the second decellularization step, and the secondary decellularization step. In particular, it can be seen that the DNA removal efficiency is significantly reduced when the second decellularization step or the secondary decellularization step was omitted. Thus, it can be confirmed through the above experiment that performing each step sequentially was more effective in DNA removal.

### Experimental Example 5

A multi-step decellularized protein extract was prepared using the same method as in Preparation Example 1, but the tissue was treated by changing the concentration of sodium hydroxide contained in the treatment solution in the primary decellularization step to 0.005 M, 0.1 M, 0.2 M, and 0.3 M.

During the experiment, the state of the tissue during and after treatment with the second decellularization step (second process) was photographed and is shown in FIG. 5. In addition, the content of DNA contained in 1 mg of the finally obtained protein extract was measured by using the same method as in Experimental Example 3, and the yield of the protein extract was calculated by expressing the content of the finally obtained protein extract as a percentage value relative to the content of the initially introduced tissue, and the H&E-stained photograph was taken and shown in FIG. 5.

Referring to FIG. 5, it is confirmed that when the concentration of NaOH is 0.05 M, the yield is good, but the DNA content is high and there are cell nuclei that are not removed within the tissue, so it can be seen that the decellularization efficiency is low.

On the other hand, it can be confirmed that when treated with 0.1 M NaOH, the structure of the tissue is well maintained in the second decellularization step, the DNA content after the final treatment is very low at 0.6 ng/mg, and the yield is high at 20.8%.

When treated with 0.2 M NaOH, the DNA content was low, but some of the tissue was disintegrated during the primary decellularization step, so the final yield was less than half that of when treated with 0.1 M NaOH. When treated with 0.3 M NaOH, the tissue was almost dissolved, so it was impossible to obtain the tissue.

Therefore, from the above experimental results, it can be confirmed that in order to obtain a protein extract with an excellent yield and sufficient removal of DNA in the tissue without tissue disintegration or dissolution in the primary decellularization step, the concentration of NaOH contained in the treatment solution in the primary decellularization step, particularly in the second decellularization step, was more than 0.05 M and less than 0.2 M.

### Experimental Example 6

A multi-step decellularized protein extract was prepared using the same method as in Preparation Example 1, but in the primary decellularization step, when the concentration of sodium hydroxide contained in the treatment solution was 0.005 M and 0.1 M, the concentration of Dnase was changed from 0.0001 to 0.003 wt % to treat the tissue. The DNA content ratio and the yield of the protein extract of each finally obtained protein extract were calculated, and the results are shown in Table 2.

TABLE 2

| NaOH | 0.05 | | | 0.1 | | |
|---|---|---|---|---|---|---|
| concentration | | | | | | |
| DNase | 0.0028 | 0.0014 | 0.0007 | 0.0002 | 10.0002 | 0.0001 |
| concentration | | | | | | |
| (wt %) | | | | | | |
| DNA | 25.7 | 38.0 | 54.7 | 0.2 | 0.3 | 0.6 |
| concentration | | | | | | |
| (ng/mg) | | | | | | |
| Yield (%) | 20.3 | 20.7 | 19.9 | 21.0 | 21.4 | 20.8 |

Referring to the results in Table 2 above, it can be seen that when the concentration of NaOH was constant, the concentration of DNA in the final protein extract decreases as the concentration of DNase increases. However, it can be confirmed that when the concentration of NaOH was 0.1 M, the DNA concentration in the final protein extract obtained was actually lower even though treatment with DNase was performed at a concentration several to several tens of times lower than when the concentration was 0.05 M, and the difference in yield was not significant.

When the concentration of NaOH is 0.05 M, the DNA removal efficiency is significantly lower than when it is 0.1 M, so it can be seen that the concentration of NaOH in the treatment solution used in the primary decellularization step is preferably greater than 0.05 M.

### Preparation Example 2

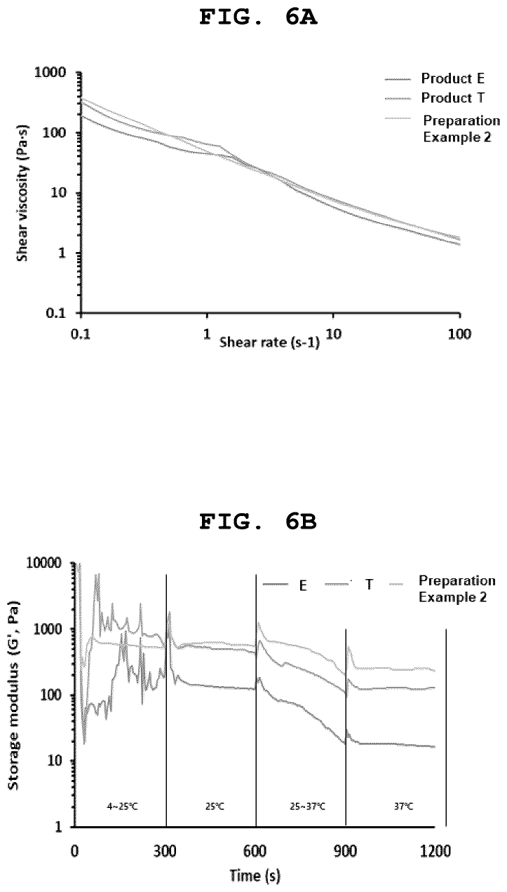

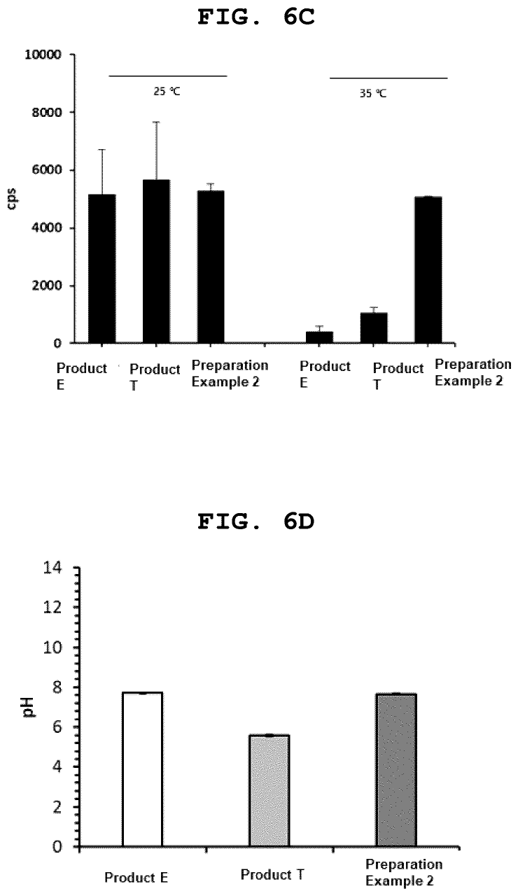

A topical wound dressing was prepared by mixing 55 g of PEG having a molecular weight of 400, 25 g of PEG having a molecular weight of 4,000, 0.35 g of the protein extract according to Preparation Example 1, and 4 g each of 1,2-hexanediol, ethylhexylglycerin, and sodium hyaluronate, and 7.65 g of purified water. The results of measuring the basic properties of the topical wound dressing thus prepared, such as viscosity, storage modulus, and pH, are shown in FIG. 6.

For comparison, the basic properties were measured under the same conditions using existing commercially available wound dressings from other manufacturers, E and T products.

As confirmed by the change in viscosity according to the change in shear rate in FIG. 6A, the topical wound dressing according to Preparation Example 2 exhibited a typical shear thinning phenomenon in which the shear viscosity decreased as the shear rate increased, similar to existing commercialized products of other manufacturers (Products E and T). However, from FIG. 6B, the storage modulus value was confirmed to have the highest value in all temperature ranges compared to Products E and T. This is believed to be because the topical wound dressing according to Preparation Example 2 contains PEG having a relatively high molecular weight.

In particular, as confirmed in FIG. 6C, when comparing viscosity values measured at room temperature (25° C.) with those measured at 35° C., which is similar to body temperature, in the case of the existing E and T products, the viscosity decreases rapidly as the temperature increases, but in the case of the topical wound dressing of Preparation Example 2, it can be seen that it has substantially the same viscosity value with almost no viscosity change. This is understood to be because PEGs having low molecular weight and high molecular weight are mixed to form a stable hydrogel.

The pH measurement results in FIG. 6D showed that the pH was in the neutral region with no significant difference from existing commercial products.

### Experimental Example 7

To confirm the effect of the protein extracts used in the topical wound dressing of the present disclosure, an in vitro tests were performed using the test groups in Table 3 below.

TABLE 3

| Group | Hydrogel | Cell |
|---|---|---|
| G1 | Atelocollagen | Fibroblast |
| | | (Lonza) |
| G2 | Atelocollagen + 0.35% protein | Fibroblast |
| | extract | (Lonza) |
| G3 | Atelocollagen + 1% protein extract | Fibroblast |
| | | (Lonza) |

### (1) Hydrogel Production

As shown in Table 3 above, the protein extracts (0, 0.35, and 1 w/v %, respectively) obtained in Preparation Example 1 and fibroblasts (1×10⁵ cells/ml) were mixed with atelocollagen, and then gelled at 37° C. for 30 minutes to produce a hydrogel sample.

### (2) CCK-8 Assay

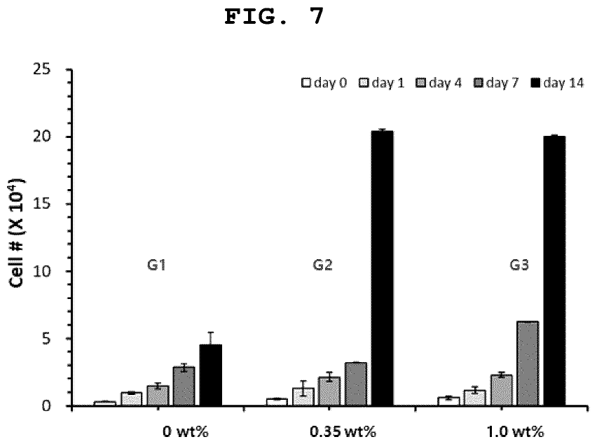

Cell Count Kit-8 (CCK-8, Dojindo, Japan) was used to measure cell proliferation rate. Each hydrogel sample prepared was cultured and analyzed on days 0, 1, 4, 7 and 14 after culture. A solution mixed with cck-8 solution and culture solution at a ratio of 1:10 was sampled and treated, and the absorbance was measured at a wavelength of 450 nm for 2 h at 37° C. The absorbance thus measured was converted into the number of cells using a standard curve using cells, and the results are summarized in FIG. 7.

As confirmed in the results in FIG. 7 above, the G2 and G3 samples containing protein extract showed enhanced cell proliferation effect in all test intervals (day 1-day 14) compared to the G1 sample without protein extract.

### (3) L/D Staining

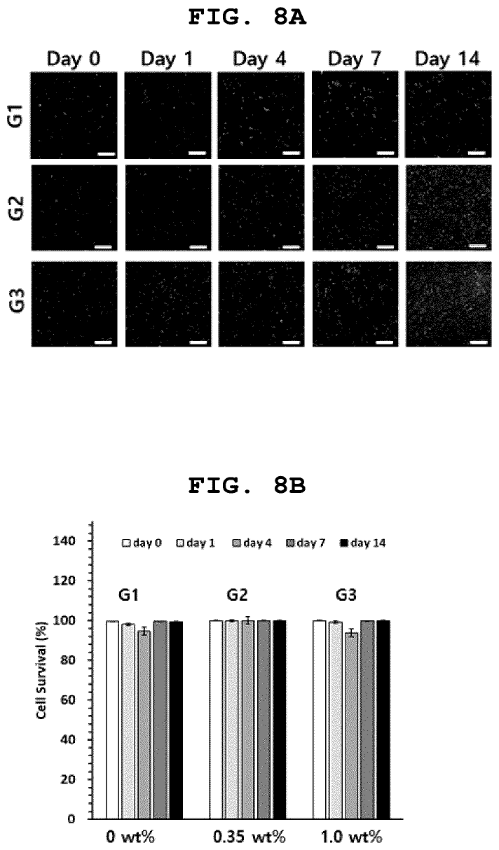

To measure cell viability in the hydrogels, experiments were performed on G1 to G3 using the Live/Dead assay kit (Invitrogen). After culturing each prepared hydrogel sample, analysis was performed on days 0, 1, 4, 7, and 14, and the culture solution containing 2 μM calcein AM and 4 μM EthD-1 was treated at each time point and observed using a fluorescence microscope (see FIG. 8A). The cell viability was calculated according to Equation 2 below and summarized in FIG. 8B.

$$Cell survival rate ( \% ) = [ ( Live cell number ) \text{} / ( Live cell number + Dead cell number ) ] \times 100 \qquad ( Equation 2 )$$

It can be seen from the results in FIGS. 8A and 8B that the cell viability in the hydrogel of the sample containing protein extract is relatively higher than that of the sample containing no protein extract.

### (4) Scratch Test

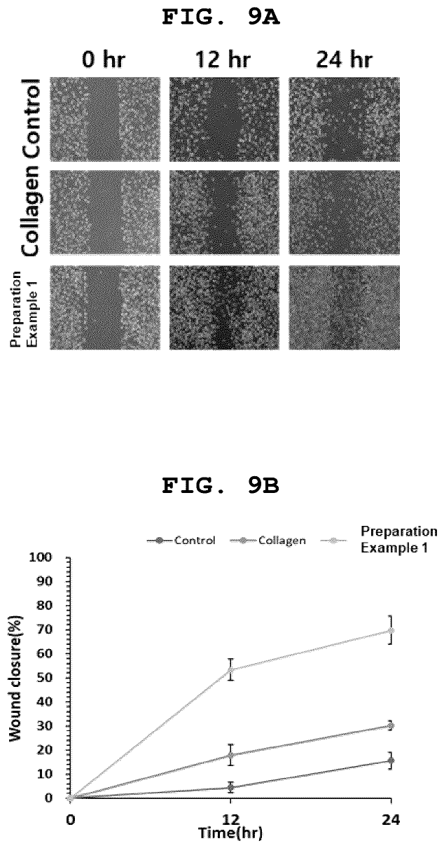

L929 cells were cultured in 6 wells at a concentration of 2×10⁵ cells/well, and a scratch was made across the center of the wells using a pipette tip. Here, the culture solution used was α-MEM culture solution mixed with 2% FBS. The wells where scratches were created were treated with the respective eluted culture solutions (each material was immersed in α-MEM culture solution mixed with 2% FBS (Fetal Bovine Serum) at a concentration of 4 g/20 ml and eluted for 24 hours under conditions of 37° C. and 5% CO₂) as shown in Table 4 below, and experiments were carried out by culturing cells on each eluted solution. After culturing, the scratched area was photographed every 0, 12, and 24 hours to analyze the rate at which the scratches were filled (see FIG. 9A). The rate at which the scratches were filled (wound closure rate) was determined by using Equation 3 below and summarized in FIG. 9B.

TABLE 4

| | Eluted | Elution | Elution | Elution |
|---|---|---|---|---|
| Group | material | ratio | solution | condition |
| Control | Culture | 4 g/20 ml | α-MEM + | 24 hours at |
| | solution | | 2% FBS | 37° C. and 5% |
| Collagen | Collagen | | | CO₂ |
| Protein | Protein | | | condition |
| extract | extract | | | |
| (Preparation | | | | |
| Example 1) | | | | |

$$Wound closure rate ( \% ) = [ ( Wound area at {t}_{0} - Wound area at {t}_{final} ) / ( Wound area at {t}_{0} ) ] \times 100 \qquad ( Equation 3 )$$

It can be confirmed from FIGS. 9A and 9B that when the protein extract prepared in Preparation Example 1 was used, the generated scratch was effectively filled compared to the untreated control group and simple collagen, which means that the protein extract according to the present disclosure may effectively act on topical wounds.

### Experimental Example 8

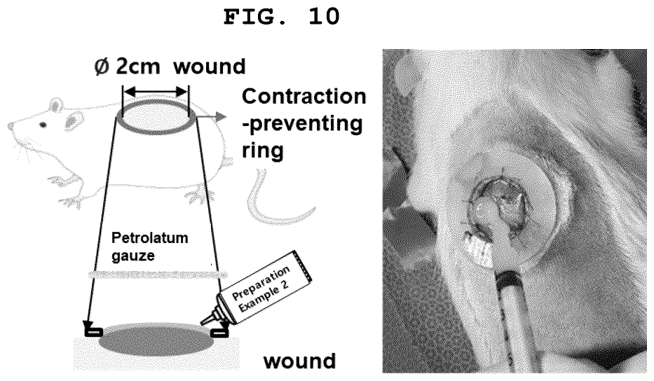

In order to confirm the effect of the topical wound dressing according to the present disclosure, the topical wound dressing according to Preparation Example 2 was applied to the topical wound area formed in a rat, and then protected using a petrolatum gauze (see FIG. 10). For comparison, the same topical wound was formed and covered with a petrolatum gauze without any treatment and used as a control.

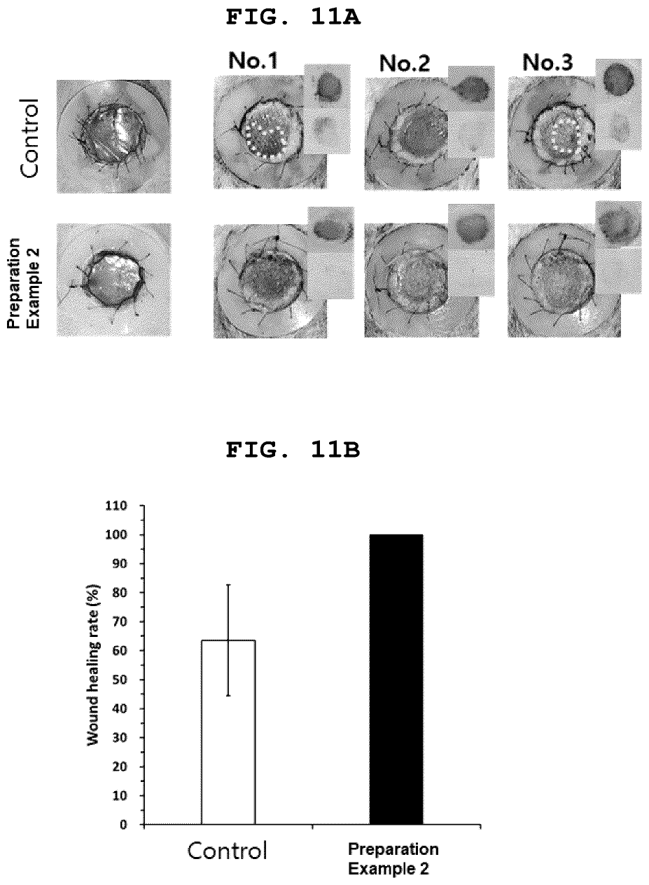

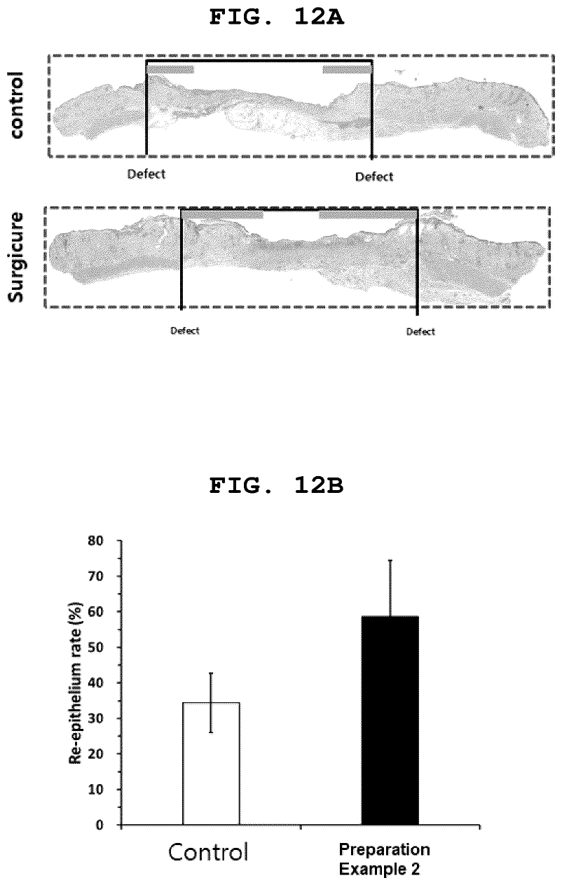

In these acute wound animal models, wound healing rate (%), re-epithelialization rate (%) and angiogenesis (%) were determined using the following Equation, and the respective photographs and results are shown in FIGS. 11A to 13B.

First, the wound healing rate presented in FIGS. 11A and 11B were confirmed by visually observing (taking photographs) the wound area of each treatment group on day 13 after treating the wound area to confirm the area where the wound area was still exposed. The gauze was placed on the wound site to reconfirm the presence of blood, and the total area of the wound and the area that remained unhealed were calculated by image analysis using Equation 4 below.

$$Wound healing rate ( \% ) = [ ( initial defect - remaining defect ) / ( initial defect ) ] \times 100 \% \qquad ( Equation 4 )$$

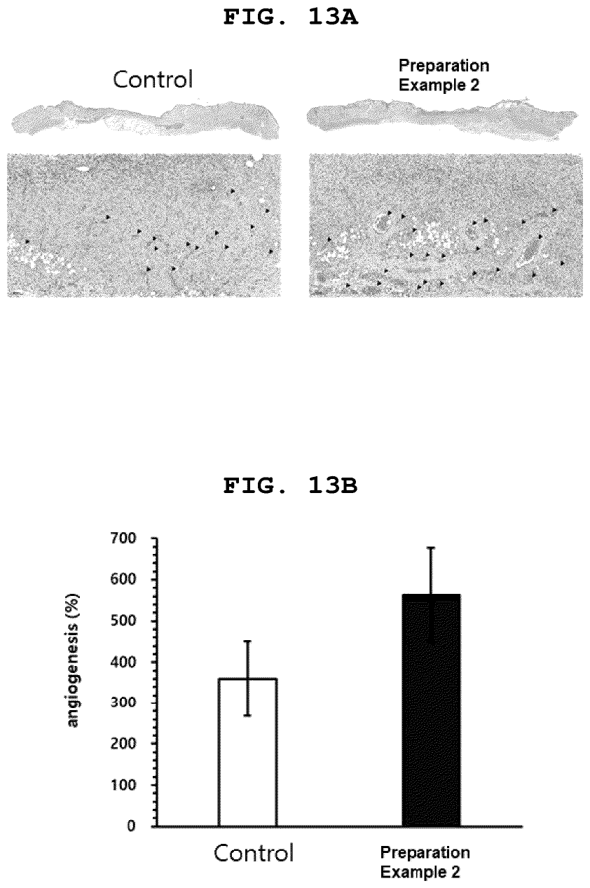

Next, samples were collected and histological assays were performed. To analyze the re-epithelialization and neovascularization rate, the central part of the wound (diameter of the circular wound in FIG. 12A) was collected to create a paraffin block, which was then sectioned and stained with hematoxylin and eosin staining (H&E staining).

H&E-stained tissue was observed and analyzed, and the length of the entire wound (defect length) and the length of the part where re-epithelialized had occurred (re-epithelium length) were measured. Based on the measured length, it is calculated using Equation 5 below, and the result are as shown in FIG. 13B.

$$Re - epithelium rate ( \% ) = [ ( re - epithelium length ) / ( initial defect length ) ] \times 100 \% \qquad ( Equation 5 )$$

In addition, to confirm the extent of neovascularization, H&E-stained tissues were observed and the number of blood vessels present per unit area was counted. The number of blood vessels counted per unit area was compared with the number of blood vessels present in normal tissue, and angiogenesis (%) was calculated by Equation 6 below, and the results are as shown in FIG. 13B.

$$angiogenesis ( \% ) = [ ( number of regenerated blood vessel in regenerated area ) / ( number of blood vessel in normal tissue ( skin dermis ) ) ] \times 100 \% \qquad ( Equation 6 )$$

It can be seen from FIGS. 11A to 13B that the topical wound dressing according to the present disclosure contains a protein extract obtained through a multi-step decellularization process, and as expected from the in vitro results of Experimental Example 7 above, there are remarkable wound healing rate, re-epithelialization rate, and angiogenesis effects in an actual acute wound animal model.

The present disclosure is not limited to the specific embodiments and descriptions described above, and various modifications may be made by anyone having ordinary skill in the art to which the present disclosure belongs without departing from the spirit of the present disclosure claimed in the claims, and such modifications will fall within the scope of protection of the present disclosure.

### Industrial Availability

The present disclosure relates to a hydrogel-based wound dressing containing a protein extract obtained through a multi-step decellularization process, and has industrial applicability because it can provide a hydrogel-based topical wound dressing containing polyethylene glycol (PEG), white petrolatum, 1,2-hexanediol, ethylhexylglycerin, sodium hyaluronate, a protein extract, and purified water.

1. A hydrogel-based topical wound dressing, comprising polyethylene glycol (PEG), 1,2-hexandiol, ethylhexylglycerin, sodium hyaluronate, a protein extract, and purified water,
wherein the protein extract is prepared by a pretreatment step of preparing and pretreating tissue from a mammal other than a human; a first inactivation step of inactivating a virus contained in the pretreated tissue using alcohol; a decellularization step of removing cells from the virus-inactivated tissue; and a second inactivation step of inactivating a virus contained in the decellularized tissue using acid,
wherein the DNA content of the tissue subjected to the second inactivation step is 50 ng/mg or less,
wherein a percentage reduction (L) of the elastin content of the tissue subjected to the pretreatment step or the second inactivation step is more than 0 and 20% or less,
wherein the decellularization step comprises: a primary decellularization step of removing cells from the virus-inactivated tissue using a basic aqueous solution; and a secondary decellularization step of removing cells by enzymatically treating the primary decellularized tissue, and
wherein the primary decellularization step comprises: a first decellularization step performed using a mixture of n-PrOH and NaOH; and a second decellularization step performed using an aqueous sodium hydroxide solution of more than 0.05 M and less than 0.2 M.

2. The hydrogel-based topical wound dressing of claim 1, wherein the hydrogel-based topical wound dressing comprises
11 to 95 wt % of polyethylene glycol, 0.1 to 10 wt % of 1,2-hexandiol, 0.01 to 10 wt % of ethylhexylglycerin, 0.001 to 10 wt % of sodium hyaluronate, 0.01 to 10 wt % of a protein extract, and residual purified water.

3. The hydrogel-based topical wound dressing of claim 1, wherein the tissue is at least one of mammal-derived blood vessels, ligaments, and tendons.

4. The hydrogel-based topical wound dressing of claim 1, wherein the secondary decellularization step is performed using DNase.

5. The hydrogel-based topical wound dressing of claim 1, wherein the polyethylene glycol (PEG) is a mixture of PEG having a weight average molecular weight of 400 and PEG having a weight average molecular weight of 4,000 in a weight ratio of 2.2:1.

6. The hydrogel-based topical wound dressing of claim 1, wherein the viscosity measured at 25° C. is substantially the same as the viscosity measured at 35° C.

7. The hydrogel-based topical wound dressing of claim 1, wherein the protein extract contains collagen and elastin.

8. The hydrogel-based topical wound dressing of claim 1, wherein the 1,2-hexanediol and ethylhexylglycerin are replaced by at least one selected from the group consisting of chlorhexidine gluconate, chlorhexidine acetate, chlorhexidine hydrochloride, silver sulfadiazine, povidone iodine, benzalkonium chloride, purazine, idocaine, hexachlorophene, chlortetracycline, neomycin, penicillin, gentamicin, gentamicin sulfate, acrinol, ciprofloxacin, benzalkonium chloride, fusidic acid, neomycin sulfate, ofloxacin, and tobramycin.
